(19) United States
(12) Patent Application Publication
(10) Pub. No.: US 2011/0124860 A1
(43) Pub. Date: May 26, 2011

(54) PROCESS FOR THE SYNTHESIS OF 3,6-DIHYDRO-1,3,5-TRIAZINE DERIVATIVES

(76) Inventors: Stéphanie Aubourg, Saint Jean de Braye (FR); Bernard Boudet, Pithiviers (FR); Daniel Cravo, Sartrouville (FR); Matthias Helmreich, Heidelberg (DE)

(21) Appl. No.: 12/736,856

(22) PCT Filed: Apr. 24, 2009
(86) PCT No.: PCT/EP2009/002997

§ 371 Date: Feb. 8, 2011

(30) Foreign Application Priority Data
May 23, 2008 (EP) 08009483.2

(51) Int. Cl. C07D 251/18

(52) U.S. Cl. 544/208; 544/204

(57) ABSTRACT

A new process for the synthesis of 3,6-dihydro-1,3,5-triazine derivatives for the treatment of disorders associated with insulin-resistance syndrome.

3,6-Dihydro-1,3,5-triazine derivatives show pharmacological properties in the treatment of pathological conditions associated with the insulin-resistance syndrome. Several patents describe the preparation of 3,6-dihydro-1,3,5-triazine derivatives. For example, in U.S. Pat. No. 3,287,366 the synthesis of dihydro-triazine bearing the following structure is described:

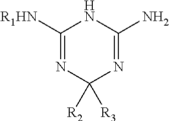

The synthesis involves the reaction of a mono-substituted bisguanidine and an aldehyde or ketone in presence of an acid at elevated temperatures.

Patent JP48064088 describes the synthesis of dihydro-triazines bearing the following structure:

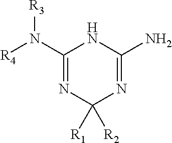

The analogous synthesis also involves heating under acidic condition.

Patent JP54014986 describes the synthesis of dihydro-triazines bearing the following structure:

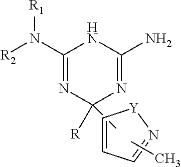

Similarly, this method requires heating under acidic conditions.

Patent application WO 01/55122 describes the synthesis of dihydro-triazines of the following structure:

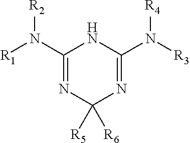

The synthesis is directed to the reaction of mono-substituted bisguanidines and an acetal, hemiacetal, ketal, hemiketal, aldehyde, or ketone in presence of an acid at elevated temperatures.

Common to the published procedures is the requirement of elevated temperature, which may require refluxing conditions or high pressure if low boiling point starting materials are employed as well as the use of an acid. The invention had the object of finding a new reaction under ambient conditions while only inexpensive starting materials are used.

This would save energy and improve the safety of the process.

Unexpectedly, it has been found, that compounds of formula I can be prepared either in absence of a base or in presence of a base selected from the group K₂CO₃, NaHCO₃, NaOMe, Na₂CO₃, piperidine, morpholine, preferably at temperatures between −5° and 80° C. under ambient pressure.

More preferably, the reaction is carried out at temperatures between −5° and 60° C. under ambient pressure.

The invention relates to a process for the preparation of compounds of the formula I

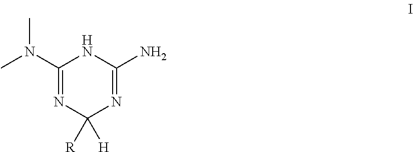

in which
R is methyl, phenyl, 4-hydroxy-phenyl or 4-methoxyphenyl
and pharmaceutically salts, tautomers and stereoisomers thereof,
characterised in that a compound of the formula II

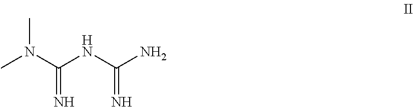

and the salts thereof
is reacted with a compound of the formula III

R—CHO III

in which R is as defined above,
in a polar solvent or solvant mixture in presence or absence of an anorganic and/or organic base, wherein the base is selected from the group
K₂CO₃, NaHCO₃, NaOMe, Na₂CO₃, piperidine, morpholine.

The solvent may be chosen from

water, methanol, ethanol, isopropanol, n-butanol, 2-butanol, i-butanol, t-butanol, N,N-dimethyl formamide or any combination of solvents.

The base particularly preferred is NaOMe or piperidine.

The solvent particularly preferred is methanol, isopropanol or a mixture of water and methanol.

The concentration of the compound of formula II ranges from 0.1 mol/L to 4 mol/L. The concentration of the compound of formula III ranges from 1 equivalent to 10 equivalents to the compound of formula II.

The base ranges from 0.5 equivalents to 10 equivalents to the compound of formula II.

The compounds of formula I also mean their solvates and their pharmaceutically usable derivatives.

The term “solvates of the compounds” is taken to mean adductions of inert solvent molecules onto the compounds which form owing to their mutual attractive force. Solvates are, for example, mono- or dihydrates or alcoholates.

The term “pharmaceutically usable derivatives” is taken to mean, for example, the salts of the compounds according to the invention and so-called prodrug compounds.

The term “prodrug derivatives” is taken to mean compounds of the formula I which have been modified with, for example, alkyl or acyl groups, sugars or oligopeptides and which are rapidly cleaved in the organism to form the active compounds according to the invention.

These also include biodegradable polymer derivatives of the compounds according to the invention, as described, for example, in Int. J. Pharm. 115, 61-67 (1995).

Formula I also embraces the tautomeric forms of the compounds.

Tautomeric forms of the compound of formula I in which R is methyl:

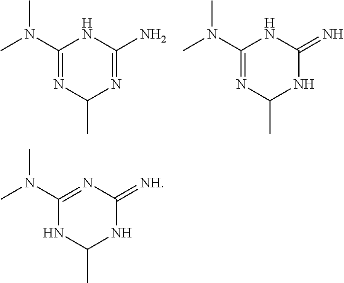

A preferred process for the preparation of compounds of formula I is related to compounds

in which
R is methyl or 4-hydroxyphenyl; most preferably R is methyl.

If N,N-dimethylbiguanide hydrochloride is used as educt the process is carried out in presence of a base.

If N,N-dimethylbiguanide (base) is used as educt the process is carried out in absence of a base.

Most preferably the reaction is carried with NaOMe as base in methanol at temperatures between −5° and 20° C.

Most preferred is the process for the preparation of the compound of formula I

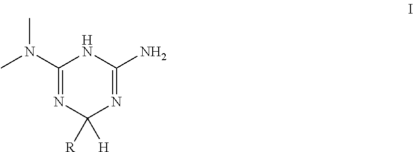

in which
R is methyl
and pharmaceutically salts, tautomers and stereoisomers thereof,
characterised in that a compound of the formula II

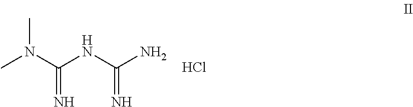

is reacted with a compound of the formula III

R—CHO III

in which R is as defined above,
in a polar solvent or solvant mixture in presence of an anorganic and/or organic base, wherein the base is selected from the group
K₂CO₃, NaHCO₃, NaOMe, Na₂CO₃, piperidine, morpholine.

There is an example giving further detail on the invention, but the invention is not limited within the example.

### EXAMPLE 1

### Preparation of 2-amino-3,6-dihydro-4-dimethylamino-6-methyl-1,3,5-triazine

A suspension of 10 g (60 mmol) N,N-dimethylbiguanide hydrochloride in 50 ml methanol is cooled to 0 to −5° C. 5.3 g (120 mmol) and 10.9 g (60 mmol) NaOMe solution (30% in methanol) are added. HPLC after 20 hours shows conversion to the desired product;

¹H NMR (300 MHz, CDCl₃) δ 1.37 (d, J=6.0 Hz, 3H), 2.98 (s, 6H), 3.46 (s, 1H), 4.83 (q, J=6.0 Hz, 1H). ¹³C NMR (300 MHz, CDCl₃) δ 24.5, 36.4, 63.0.

### EXAMPLE 2

### Preparation of 2-amino-3,6-dihydro-4-dimethylamino-6-(4-hydroxyphenyl)-1,3,5-triazine

A suspension of 30.42 g (0.235 mol) N,N-dimethylbiguanide, 30.2 g (0.246 mol) para-hydrobenzaldehyde and 3 ml piperidine in 300 ml isopropanol is stirred at reflux for 20 hours. Upon cooling, a solid precipitates, which is recovered and washed with cooled isopropanol; yield: 42 g 2-amino-3,6-dihydro-4-dimethylamino-6-(4-hydroxyphenyl)-1,3,5-triazine.

1. Process for the preparation of compounds of formula I 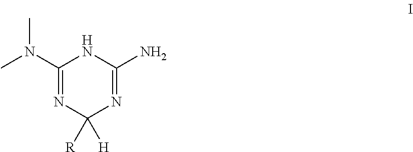
in which
R is methyl, phenyl, 4-hydroxy-phenyl or 4-methoxyphenyl
and pharmaceutically salts, tautomers and stereoisomers thereof,
characterised in that a compound of the formula II 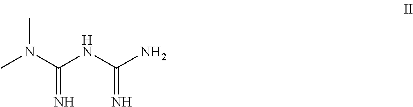
and the salts thereof
is reacted with a compound of the formula III
R—CHO III
in which R is as defined above,
in a polar solvent or solvant mixture in presence or absence of an anorganic and/or organic base, wherein the base is selected from the group
K₂CO₃, NaHCO₃, NaOMe, Na₂CO₃, piperidine, morpholine.

2. Process according to claim 1, in which R is methyl or 4-hydroxyphenyl.

3. Process according to claim 1, in which the base is chosen from the group of NaOMe or piperidine.

4. Process according to claim 1, in which the solvent is chosen from the group as single solvent or solvents mixture: water, methanol, ethanol, isopropanol, n-butanol, 2-butanol, i-butanol, t-butanol, N,N-dimethyl formamide.

5. Process according to claim 1, in which the concentration of compound of formula II is from 0.1 mol/L to 4 mol/L.

6. Process according to claim 1, in which the compound of formula III is from 1 equivalent to 10 equivalents to the compound of formula II.

7. Process according to claim 1, in which the base is from 0.9 equivalent to 10 equivalents to the compound of formula II.

8. Process according to claim 1, in which the compound of formula III is acetaldehyde.

9. Process according to claim 1, in which the reaction is performed at temperatures between −10 and 80° C. under ambient pressure.

10. Process according to claim 1, in which the reaction is performed at room temperature at temperatures between −5 and 60° C. under ambient pressure.

11. Process according to claim 1, in which the reaction is performed at room temperature at temperatures between −5 and 20° C. under ambient pressure.
